(19) United States
(12) Patent Application Publication
(10) Pub. No.: US 2007/0023710 A1
(43) Pub. Date: Feb. 1, 2007

(54) Closed-loop control of ultraviolet (UV) sterilization systems

(75) Inventors: Curtis Tom, San Mateo, CA (US); Mathieu Herbette, Sunnyvale, CA (US); Orion Weihe, Fremont, CA (US)

Correspondence Address: BUCHANAN, INGERSOLL & ROONEY PC, POST OFFICE BOX 1404, ALEXANDRIA, VA, 22313-1404, US

(73) Assignee: Amarante Technologies, Inc., Santa Clara, CA (US)

(21) Appl. No.: 11/483,267

(22) Filed: Jul. 6, 2006

Related U.S. Application Data
(60) Provisional application No. 60/697,630, filed on Jul. 8, 2005.

(51) Int. Cl. A61L 2/10; A61L 2/24

(52) U.S. Cl. 250/504 R; 422/62; 422/24; 422/3

(57) ABSTRACT

Apparatus and methods for controlling an ultraviolet (UV) sterilization system that has one or more UV sources to achieve proper sterilization with minimal time and usage of power for the UV sterilization system. The UV sources are operated according to a set of control parameters including intensity of the UV light energy and an exposure time. The device may include: one or more sensors that are configured to measure UV light energy emitted by the UV sources and develop one or more signals; and a microcontroller that has an access to information of UV energy doses for various types of microorganisms. The microcontroller is configured to receive the signals from the sensors, determine a set of optimum values corresponding to the set of control parameters using the signals and the information, and send the set of optimum values to the UV sources to control the UV sources.

### CROSS-REFERENCE TO RELATED APPLICATION

This application claims priority to U.S. Provisional Patent Application No. 60/697,630, filed Jul. 8, 2005, which is incorporated herein in its entirety.

### BACKGROUND

The effective removal of viable pathogenic microorganisms is essential to those who regularly come into contact with potential infectious microorganisms. Medical caregivers, such as medical doctors, dentists, etc., are frequently exposed to bodily fluids that may contain infectious microorganisms, such as bacteria, viruses, or the like. Instrumentation (including human hands) must be effectively sterilized to prevent the transmission of potentially infectious microorganisms and protect themselves from such microorganisms.

Ultraviolet (UV) light has long been used for disinfection and sterilization of organic and/or inorganic matter. For simplicity, hereinafter, the term “microorganisms” collectively refers to organic and/or inorganic matter to be sterilized. Exposure to certain ultraviolet light band wavelengths has been discovered to be an effective means for destroying microorganisms. Typically, in using this method of sterilization, the user places the object to be cleaned into a sterilization chamber (or, equivalently cleaning chamber) to expose the device or object to be cleaned to a prescribed dose of ultraviolet light.

In general, a conventional UV sterilization system determines the prescribed dose of ultraviolet energy by controlling the amount of operational interval for each UV lamp (or, equivalently exposure time). To use the conventional UV sterilization system for complete sterilization of various microorganisms that can cause potentially life threatening conditions, the intensity of UV light as well as the amount of exposure time needs to be monitored and controlled in a precise manner. Thus, there is a strong need for a technique to control UV sterilization systems based on the intensity and exposure time such that the optimum dose of UV light for each type of target microorganism can be provided with enhanced efficiency and reliability.

### SUMMARY

According to one embodiment, a device for controlling an ultraviolet (UV) sterilization system having one or more UV sources operated according to a set of control parameters includes: one or more sensors that are configured to measure LTV light energy emitted by the UV sources and develop one or more signals; and a microcontroller having an access to information of UV energy doses for various types of microorganisms, and configured to receive the signals from the sensors, determine a set of optimum values corresponding to the set of control parameters using the signals and the information, and send the set of optimum values to the UV sources to control the UV sources.

These and other features, aspects and advantages of the present invention will become better understood with reference to the following drawings, description and claims.

### BRIEF DESCRIPTION OF THE DRAWINGS

FIG. 1 shows a perspective view of a UV sterilization device in accordance with one embodiment.

FIG. 2 shows a schematic diagram of a movable UV sensor used in the device of FIG. 1.

FIG. 3 shows a functional diagram of a UV sterilization system having a closed-loop control module in accordance with another embodiment.

FIG. 4 shows a flow chart illustrating a process for sterilizing targets by use of the UV sterilization device in FIG. 1 in accordance with yet another embodiment.

### DETAILED DESCRIPTION OF THE DISCLOSURE

The following detailed description is of the best currently contemplated modes of carrying out the invention. The description is not to be taken in a limiting sense, but is made merely for the purpose of illustrating the general principles of the invention, since the scope of the invention is best defined by the appended claims.

It must be noted that, as used herein and in the appended claims, the singular forms “a”, “and”, and “the” include plural referents unless the context clearly dictates otherwise. Thus, for example, reference to “a sensor” includes one or more sensors and equivalents thereof known to those skilled in the art, and so forth.

Certain embodiments include UV sterilization systems that are based on a closed-loop control technique for controlling the intensity of UV light and/or exposure time to achieve proper sterilization with minimal time and usage of power for the systems. Unlike existing UV sterilization systems merely based on exposure time control, the UV sterilization systems of certain embodiments may provide the optimum dose of the UV light and thereby to enhance the efficiency and reliability of the systems.

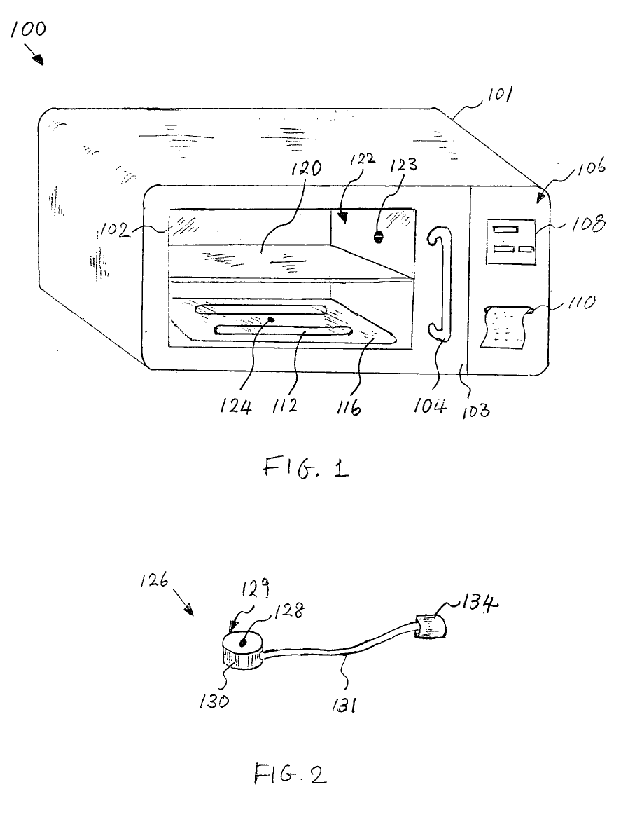

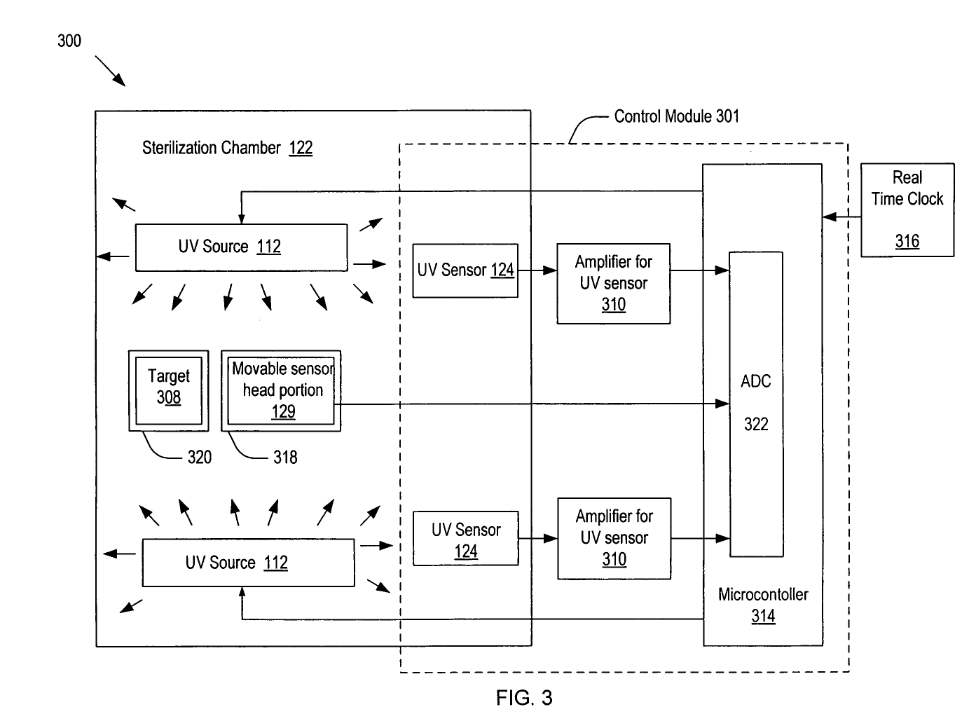

FIG. 1 is a schematic diagram of a UV sterilization device 100 in accordance with one embodiment. As depicted, the device 100 may have an appearance of a small tabletop appliance and use UV radiation to sterilize targets disposed therewithin. The targets to be sterilized may be of any suitable size and shape, and include tools and instruments used in typical healthcare facilities. The device shown at 100 may include multiple UV sources or lamps 112 monitored and controlled by a microcontroller to provide a sufficient dosage of UV radiation to maximize sterilization efficacy. More detailed description of the microcontroller will be given in conjunction with FIG. 3. The entire device 100 may be housed in an enclosure 101 designed to prevent leakage of UV light and to protect the electronic and mechanical subsystems from various types of damages.

The outer enclosure 101 of the UV sterilization device 100 may be constructed of material, such as stainless steel, to withstand harsh cleaning and disinfecting chemicals typically found during operation of the device 100. The device 100 may include a sterilization chamber 122 for disposing the targets to be sterilized therein. The front side of the enclosure 101 may include a door 103 with a UV blocking window 102 to allow the user or operator to view the contents or targets within the sterilization chamber 122. The window 102 may be constructed of an optically clear material, such as polycarbonate which will absorb UV in the range of 200 to 400 nm. The door 103 may have a handle 104 for the user to unlatch and open the door 103. The latch mechanism may contain a series of mechanical switches that function as a safety interlock to inform the microcontroller the door 103 is open (or not properly closed) and to de-energize the UV sources 112 thus preventing accidental UV exposure to the operator. The front panel 106 may contain a LCD/touch panel 108 that allows the user to control the device 100, such as to program sterilization times, perform self diagnostics, and to start (or abort) the sterilization cycle. The lower area of the front panel 106 may have a thermal printer 110 that provides a hardcopy of the sterilization information including time, date, exposure/sterilization cycle times, and pass/fail status. The lower area of the front panel 106 may also contain a door to allow the user to reload the printer 110 with paper as needed.

The sterilization chamber 122 may include two sets of UV lamps 112 respectively located on the chamber floor and ceiling, where each set of UV lamps includes two UV bulbs. For simplicity, the two lamps located on the ceiling of the chamber 122 are not shown in FIG. 1. These high output UV lamps 112 may be designed to emit UV radiation of the “C” band with peaks in the 250 to 260 nm range. The lamps 112 may start instantly and have a long service life. To protect the lamps 112 from fluid ingress, two UV transmissive windows 116 may be respectively placed in front of the two sets of UV lamps 112 on the floor and ceiling of the chamber forming a sealed compartment. The windows 116 may be made from UV transmissive materials, such as quartz. A shelf 120 located midway between the bottom and top of the chamber may be designed to support the targets to be sterilized and permit equal exposure to UV from the top and bottom UV sources 112. To aid in uniform exposure, the entire inner surface of the chamber 122 may be designed to be highly reflective to UV. The shelf 120 may be also made from a UV transmissive material, such as quartz, to permit the UV radiation from the lower source to irradiate the bottom of the targets.

Located within banks of UV sources 112 may be U sensors 124 which monitor the output of the UV sources 112 during each sterilization cycle. As the lamps 112 may age, the UV output will diminish over time and the presence of the UV sensors 124 may allow the microcontroller to compensate the decrease in UV intensity by increasing the exposure time during each sterilization cycle thereby to maintain sterilization efficacy.

In addition to the UV sensors 124 that are fixed at predetermined locations, there may be a third sensor that can be positioned at any location within the chamber 122. FIG. 2 shows a schematic diagram of the movable sensor unit 126. This sensor unit 126 may include: a sensor head portion 129 that includes a UV sensor 128 and a puck 130 housing the UV sensor 128, the puck 30 optionally containing electronic circuitry to amplify the signal from the UV sensor 128 and being formed from a plastic material, such as Teflon; an umbilical cord 131 having multiple wires and a silicone outer jacket, one end of the umbilical cord 131 being coupled to the UV sensor 128 or optionally to the amplifying electronic circuitry contained in the puck 130; and an electrical connector 134 attached to the other end of the umbilical cord 131 and adapted to mate a receptacle 123 on the side wall of the chamber 122 and to transmit signals from the UV sensor 128 to the microcontroller. The electrical connector 134 may be imperious to UV radiation and common disinfection and cleaning chemicals. The connector 134 and the mating receptacle 123 may be commonly made of engineered polymers, such as VICTREX® PEEK™ polymer or Ultem™ polymer, that are used in various medical devices.

It is common practice in the healthcare industry to sterilize surgical tools and other items in sealed pouches so that once sterilized, these items can be stored in a non-sterile environment for later use. The UV sensor 128 and the puck 130 may be contained in a pouch in the same way as targets so that the microcontroller can take into account the UV absorption by the pouch and compensate the absorption by increasing the exposure time.

Referring now to FIG. 3, FIG. 3 is a functional diagram of a UV sterilization system shown at 300 having a closed-loop control module 301 in accordance with another embodiment. The system shown at 300 may be an integral part of the device 100 of FIG. 1. As depicted in FIG. 3, the control module 301 may include: two UV sensors 124 for measuring UV light intensity emitted by two sets of UV sources 112 located on the floor and ceiling of the chamber 122; two amplifiers 310 for respectively amplifying signals from the two sensors 124; and a microcontroller 314 for receiving signals from the amplifiers 310, developing control signals, and sending the developed signals to the UV sources 112. Each of the control signals may include an instruction for adjusting the light intensity and/or exposure time. The control module 301, which may be embedded in the device 100, may be also coupled to and receive signals from the sensor head portion 129 of the UV sensor unit 126. The UV light sources 112 may be mercury lamps that emit UV light at a wavelength of 254 nm. For simplicity, only three sensors and two amplifiers are shown in FIG. 3. However, it should be apparent to those of ordinary skill that the present disclosure may be practiced with other suitable number of sensors and amplifiers.

As discussed above, the interior of the sterilization chamber 122 may be coated with a reflective surface which reflects the UV light to ensure that all surfaces the targets being sterilized are irradiated with a sufficient amount of the ultraviolet light, where the amount of time required for a sterilization process varies depending on the type of the target microorganisms.

The UV sensors 124 and 128 would employ one or more Silicon Carbide (SiC) UV photodiode (for example, Photonic Detector Inc. model PDU-S101) to measure the amount of UV light energy emitted by the UV sources 112. Each of the UV sensors 112 and 128 may convert the UV light energy collected thereby into a current (or, equivalently a photodiode signal) commensurate with the collected light energy. Then the signal from the sensors 124 may be sent to amplifiers 310, such as the OPA627 from Texas Instruments, so that the current generated by each sensor may be converted into a voltage commensurate the current. This voltage can be converted into a digital signal via an analog to digital converter (ADC) 322 built in the microcontroller 314. In an alternative embodiment, the ADC 322 may be positioned between the amplifiers 310 and the microcontroller 314. The microcontroller 314 may be coupled to a real time clock 316 to get elapsed time information.

As a variation, the UV sensor 124 may have a built-in amplifying circuit that can generate an amplified output signal. In another variation, each of the sensors 112 and 128 may be configured to communicate with the microcontroller 314 via a wireless connection mechanism.

To achieve sterility, a proper dose of UV light energy should be applied to the target microorganisms in the target 308. Using the signal from the sensors 124 and 128 and elapsed time information, the microcontroller 314 may adjust the exposure time of the UV sources 112 to provide the proper dose. In an alternative embodiment, the microcontroller 314 may provide the proper dose by adjusting the intensity level of the UV light energy emitted by the UV sources 112 while the exposure time is fixed. In another alternative embodiment, the microcontroller 314 may provide the proper dose by controlling both the intensity and exposure time.

As depicted in FIG. 3, each of the UV sensors 124 may collect a portion of the UV light emitted by the UV sources 112 (each sensor may face one or more UV lamps either directly, or indirectly via optical prisms or fiber optics), monitoring the status of the UV sources 112. The collected portion of the UV light may include a reflected light, an incident light or any combination thereof, depending on the geometry of the sterilization chamber 122. As the UV sources 112 may gradually diminish over the course of their service life, the signals from the UV sources 112 may indicate the status of the sources, which can be used to perform self diagnostics and inform the user of a system fault (i.e. lamp failure). The microcontroller 314 may also use the signals to adjust the exposure time providing an adequate dose of UV light energy.

In some cases, the target 308, an object to be sterilized, may be contained in a pouch 320 (or, equivalently, wrapped in a packing material). In such cases, the pouch 320 may absorb/block a portion of the UV light that otherwise may be delivered to the target 308. As the relevant variable to be measured by a sensor may be the amount of the UV light energy delivered to the target 308, the movable sensor head portion 129 may be contained in a pouch 318 in the same manner to compensate the UV energy loss by the pouch 320. The step of respectively packing the target 308 and movable sensor head portion 129 in the pouches 320 and 318 may impede the UV sterilization process. For example, each item or target 308 to be sterilized may be traditionally sealed in a pouch 320 or wrapped prior to sterilization. Wrapping the target 308 and the movable sensor head portion 129 may be done following a standardize procedure in the healthcare industry.

The microcontroller 314 may be preprogrammed (and preferably stored in a nonvolatile memory) with information of the amount of UV energy needed to sterilize the target microorganisms. During the actual sterilization cycle, the microcontroller 314 may use the signal from the UV sensors 124 to estimate the actual UV energy delivered to the target 308 and calculate, in real time, the energy being absorbed by the target microorganism. Based on this calculation, the microcontroller 314 may adjust the exposure time and send “ON” signal to the UV sources 112 until an adequate dose of UV light energy is provided.

Using the signals from the sensors 124, the microcontroller 314 may determine the state of the UV sources 112. If the UV sources 112 have degraded output, the microcontroller 314 may send “ON” signals to the UV sources 112 until the proper dose of UV light energy has been absorbed by the target microorganism to achieve sterility.

As discussed above, the control module 301 may be an integral part of the sterilization device 100 and the sensors 124 may somehow communicate with the microcontroller 314 to relay information about the sterilization chamber conditions. The microcontroller 314 may be the Atmel Mega169™ microcontroller with 16K of program memory while the ADC 322 may have 8 channels of 10 bits each.

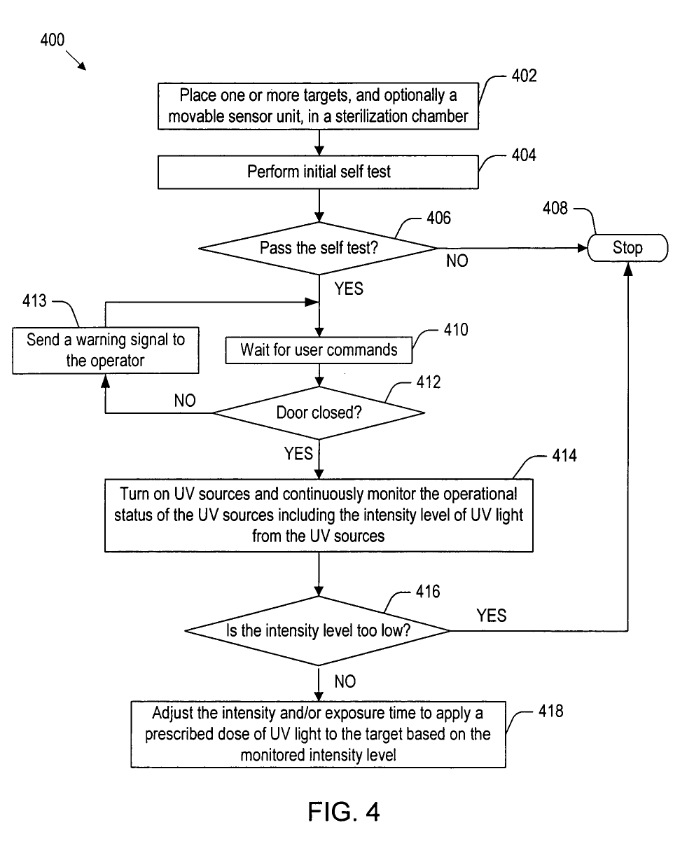

FIG. 4 shows a flow chart illustrating a process shown at 400 for sterilizing targets by use of the UV sterilization device 100 in FIG. 1. The sterilization process may begin by placing targets, and optionally the movable sensor unit 126, in the sterilization chamber 122 in a state 402. The sensor head portion 129 of the sensor unit 126 and the targets 308 may be respectively placed into the pouches 318 and 320 in advance. Next, in a state 404, the microcontroller 314 may perform initial a self test to ensure the device 100 is operational. Power supply voltage levels, UV lamps 112 and sensors 124 may be some of the items that are checked during the self test. Then, the process may proceed to a state 406. In the state 406, it is determined if the self test has successfully completed. If the device 100 fails the self test, the process stops in a state 408. Otherwise, the microcontroller 314 may enter the idle state to await user commands via the touch screen 108 on the front panel 106 in a state 410. Upon receipt of a command from the operator, in a state 412, the microcontroller 314 may first check to make sure the door latch is properly closed before energizing the UV lamps 112. If the door 103 is open, the microcontroller 314 may send a warning signal to the operator in a state 413 and the process may proceed to the state 410. Otherwise, the microcontroller 314 may turn on the UV sources or lamps 112 and begin to a real time clock that is used to track the elapsed time in a state 414. Also, in the state 414, the microcontroller 314 may continuously read the signals from the sensors 124 so that the microcontroller 314 may monitor the operational status including the intensity level of UV light from the UV sources 112. If, in a state 416, it is determined that one or more UV lamps 112 fail or have significantly diminished output and thereby the intensity level is too low to continue the cycle, the cycle may be terminated in the step 408. Otherwise, in a state 418, the microcontroller 314 may adjust the exposure time based on the monitored intensity level so that a prescribed dose is applied to the targets 308. Alternatively, in the state 418, the microcontroller 314 may adjust the intensity level and/or the exposure time depending on the type of target microorganisms.

If the sensor head portion 129 and targets 308 are placed into pouches, the microcontroller 314 may adjust the total exposure time based on the signal from the sensor 128 of the sensor head portion 129. Because most packaging materials or pouches 318 and 320 may absorb/block a portion of UV light, the sensor 128 may measure the amount of UV radiation transmitted through the pouch 318 and permit the microcontroller 314 to increase the exposure time until sufficient UV energy has been absorbed by the microorganisms in the target 308 to complete the sterilization process. The system 300 may use the two UV sensors 124 to monitor the process if the movable UV sensor 126 is not connected to the receptacle 123. If the movable UV sensor 126 is connected, it may have priority in determining the overall exposure time.

It should be understood, of course, that the foregoing relates to exemplary embodiments of the invention and that modifications may be made without departing from the spirit and scope of the invention as set forth in the following claims.

1. A device for controlling an ultraviolet (UV) sterilization system having one or more UV sources operated according to a set of control parameters, comprising:
one or more sensors, said sensors configured to measure UV light energy emitted by said UV sources and generate one or more signals; and
a control system having an access to information of a UV energy dose for one type of microorganism, and configured to receive said signals from said sensors, determine a set of optimum values corresponding to said set of control parameters using said signals and said information, and send said set of optimum values to said UV sources to control said UV sources.

2. The device of claim 1, wherein said set of control parameters include an exposure time of said UV sources and an intensity of said UV light energy.

3. The device of claim 1, wherein said microorganism is carried by a target contained in a first pouch and wherein one of said sensors may be contained a second pouch that is made of the same material for the first pouch.

4. The device of claim 1, wherein said microorganism is carried by a target wrapped by a first packing material and wherein one of said sensors may be wrapped in a second packing material, said first packing material being same as the second material.

5. The device of claim 1, wherein said signals are used to determine operational status of said sensors.

6. The device of claim 1, wherein said control system is a microcontroller.

7. The device of claim 1, wherein said ultraviolet system has a sterilization chamber that is coated with a material to reflect said UV light energy and configured to contain said sensors.

8. A device for sterilizing a target that might carry at least one type of microorganism, comprising:
a sterilization chamber having a space in which the target is to be received;
one or more ultraviolet (UV) light sources for emitting ultraviolet (UV) light energy into said space;
one or more sensor units for measuring an intensity of the UV light energy and generating one or more signals commensurate with the intensity; and
a control system having an access to information of a UV energy dose for the type of microorganism, and adapted to receive said signals from said sensor units, determine a set of optimum values corresponding to a set of control parameters using said signals and said information, and send said set of optimum values to said UV sources to control said UV sources.

9. A device as recited in claim 8, wherein said set of parameters include an exposure time of said UV light sources and the intensity of the UV light energy

10. A device as recited in claim 8, wherein said UV light sources are located on the interior surface of said chamber and wherein said sensor units include one or more UV sensors located on the interior surface of said chamber.

11. A device as recited in claim 8, wherein the target is contained in a first pouch and wherein one of said sensor units may be contained a second pouch that is made of the same material for the first pouch.

12. A device as recited in claim 8, wherein the target is wrapped by a first packing material and wherein one of said sensor units may be wrapped in a second packing material, said first packing material being same as the second material.

13. A device as recited in claim 8, further comprising an electrical receptacle formed in the interior surface of said chamber and coupled to said control system, wherein said sensor units include:
a sensor head including a puck and a UV sensor mounted therein;
an umbilical code having one end coupled to said UV sensor; and
an electrical connector couple to the other end of said umbilical code and configured to fit into said electrical receptacle.

14. A device as recited in claim 13, wherein said puck includes an amplifying circuit for amplifying said signals.

15. A device as recited in claim 8, wherein said signals are used to determine operational status of said sensor units.

16. A device as recited in claim 8, wherein said control system is a microcontroller.

17. A device as recited in claim 8, wherein the wavelength of the UV light energy ranges from 250 to 260 nm.

18. A device as recited in claim 8, wherein the interior surface of said chamber is coated with a material to reflect the UV light energy.

19. A method for sterilizing a target that might carry microorganisms by use of a device according to claim 8, comprising:
placing the target into the chamber;
turning on the UV light sources and monitoring the intensity level of the UV light energy emitted by the UV light sources; and
adjusting the intensity level and an exposure time of the UV light sources to apply a prescribed dose of UV light to the microorganisms bases on the monitored intensity level.

20. A method as recited in claim 19, further comprising:
determining an operational status of the UV light sources based on the monitored intensity level.
